(19) United States
(12) Patent Application Publication
(10) Pub. No.: US 2013/0231460 A1
(43) Pub. Date: Sep. 5, 2013

(54) METHOD AND DEVICE FOR CONCENTRATING TARGET COMPOUNDS

(71) Applicants: Christoph Erbacher, Haan (DE); Vera Holländer, Unna (DE); Markus Müller, Domagen (DE); Martin Schlumpberger, Sollingen (DE); Marion Schmidt, Langenfeld (DE); Markus Sprenger-Haussels, Mettmann (DE)

(72) Inventors: Christoph Erbacher, Haan (DE); Vera Holländer, Unna (DE); Markus Müller, Domagen (DE); Martin Schlumpberger, Sollingen (DE); Marion Schmidt, Langenfeld (DE); Markus Sprenger-Haussels, Mettmann (DE)

(73) Assignee: Qiagen GmbH, Hilden (DE)

(21) Appl. No.: 13/821,509

(22) PCT Filed: Sep. 7, 2011
(86) PCT No.: PCT/EP2011/004494

§ 371 Date: May 21, 2013

(30) Foreign Application Priority Data
Sep. 8, 2010 (EP) 10009319.4

(51) Int. Cl. G01N 1/40; C12Q 1/68

(52) U.S. Cl. CPC G01N 1/405; C12Q 1/6806
USPC 530/350; 536/23.1; 536/123.1; 422/527

(57) ABSTRACT

The present invention relates to a method for concentrating one or more target compounds in a liquid sample, a device for carrying out this method and a kit for processing a biological sample comprising such a device.

The present invention relates to a method for concentrating one or more target compounds in a liquid sample comprising said one or more target compounds dissolved in a solvent or a mixture of solvents by contacting the sample with an absorber, to a device for concentrating one or more target compounds in a liquid sample, and to a kit for processing a biological sample comprising such a device.

After extraction or purification by chromatographic procedures target compounds are usually obtained in dilute solutions. Concentration of such dilute solutions, i.e. increasing the amount of target compound per volume unit of solvent by removing at least a part of the solvent, is often necessary in order to reduce the volume of process liquid in subsequent steps or to obtain a concentration of the target compound in the sample solution that allows a reliable analysis. In general, distillation or evaporation at elevated temperatures and/or reduced pressure is the process most commonly used for concentrating a liquid sample solution.

For aqueous solutions of rather sensitive molecules, in particular biomacromolecules, such as peptides, proteins and nucleic acids, including ribonucleic acids (RNA) and desoxyribonucleic acids (DNA) which usually are present in an aqueous solution, distillation, however, is not the method of choice due to the high heat absorption capacity of water. Many biological target compounds, on the other hand, are poorly soluble in organic solvents, so that extraction or chromatography using low-boiling organic solvents does not represent an alternative.

For this reasons, methods like precipitation, ion-exchange chromatography, ultra-filtration, vacuum-dialysis, freeze-drying (lyophilisation), bind and release procedures and the like are commonly used for concentrating dilute aqueous solutions of biomacromolecules. While these methods are mild in comparison to distillation, they all require expensive and high-maintenance equipment and/or are rather time-consuming, in particular when considering concentrating a large number of samples.

In recent years, so-called super-absorbing polymers (SAP), previously mainly used as water-absorbing or humidity control agents in diapers or medical devices such as wound dressings, have been investigated in methods and devices for concentrating solutions of biological target compounds. Super-absorbing polymers are polymers possessing the ability to absorb and retain large volumes of water and aqueous solutions, e. g. an amount exceeding more than a hundredfold of their own weight. In SAPs water-absorption may be based on chemical and/or physical absorption mechanisms, for example of the absorber's micropore structure, as it is the case for example in silica gel, on physical entrapment of water via capillary forces within macropores found e.g. in polyurethane sponges, on hydration of functional groups, found e.g. in tissue paper, on essentially dissolution and thermodynamically favoured expansion of the macromolecular chains, or a combination of two or more of the before mentioned mechanisms.

For concentrating biological samples mainly water-insoluble SAPs are used that have a high molecular polymeric network, such as chemically modified starch, cellulose, polyvinyl alcohol, polyethylene oxides, cross-linked polyacrylic acid, and the like.

While SAPs have an excellent liquid-absorbing capacity, they do not absorb biological target molecules, i.e. they are highly selective concentrating agents. This is due to various effects, which also may interact. Firstly, a SAP usually represents a cross-linked polymer which upon swelling in water extends its three-dimensional network, forming pores of a certain limited size. The pore size can be controlled by the amount of cross-linker within the polymer in such a way that the pores are too small to let enter the rather large biomolecules. Secondly, SAPs usually have charged side chains whose counter-ions are able to move within the expanded three-dimensional network of the swollen polymer, but are not able to leave the polymer core. Thus, osmosis effects play a role. In addition, the SAP surface may further be modified to specifically repel a class of target molecules, e. g. by covalently attaching charged functional groups onto the SAP surface.

E. L. Cussler et al. describe the use of cross-linked, partially hydrolyzed polyacrylamide gels for concentrating solutions of high-molecular weight solutes (AIChE Journal 1984, 30 (4), 578-582). The aqueous sample solution comprising the target compound, is added to the dry polymer, which upon contact with the aqueous solution swells, preferentially absorbing the solvent, but not the target molecules. After waiting for an appropriate time to allow the gel to reach an equilibrium swelling state, the non-absorbed “raffinate”, now being a concentrated solution, can be withdrawn from the swollen polymers, for example by filtration. In additional steps, the gel may be recovered by inducing release of the retained solvent, thus shrinking the polymer.

E. Vasheghani-Farahani et al. describe a similar gel extraction process using ionic copolymers of acrylamide and N-isopropylacrylamide and homopolymers of N-isopropylacrylamide for concentrating protein solutions (Chem. Eng. Sci, 1992, 47 (1), 31-40). A similar process has also been described by M. V. Badiger et al. Chem. Eng. Sci., 1992, 47(1), 3-9.

Concentration of bovine serum albumin (BSA) using a super-absorbing polymer has been described by D. M. F. Prazeres (J. Biotechn. 1995, 39, 157-164).

In WO 2005/058453 A1 superabsorbent polymers (SAP) or superabsorptive composite materials said to be specifically adapted to the purpose of concentrating a solution of biological compounds are described. This application further describes methods of concentrating solutions of biological target compounds, either by adding a large excess of a SAP a biological sample, for example a sample of human urine or blood, allowing the liquid to get completely absorbed by the SAP or by adding a defined amount of the SAP to the liquid sample, then allowing the SAP to reach an equilibrium swelling state, afterwards recovering the remaining liquid, i.e. the concentrated sample, for example by pipetting. Using the latter procedure, in principle the amount of solvent removed can be controlled by the amount of SAP added to the sample, i.e. its weight-in quantity. A precisely determined and reproducible volume reduction, however, is only possible, if the SAP is provided in a perfectly evenly shaped form, i.e. in the form of perfectly spherical beads, as otherwise the particles within a sample would have different absorbing properties per weight unit and the amount of absorbed water could not be exactly determined by the SAP's weight-in quantity. In addition, the properties of the sample to be concentrated, that influence the absorbing properties of the SAP have to be known, such as ionic strength, pH etc.

Even though the methods described above offer the possibility of concentrating a biological sample under mild conditions without the need for expensive and high-maintenance equipment, they are still not optimized for concentrating a sample to a defined final sample volume, i.e. for removing a defined amount of solvent in a short time. The SAP-based methods described in the state of the art are in particular not suitable for high-throughput parallel processing of a multitude of biological samples.

Usually a defined amount of SAP is added to a liquid sample to remove a certain amount of solvent. Swelling of the SAP to reach an equilibrium swelling state requires a certain amount of time. In addition, the extent of swelling and the amount of water/solvent absorbed critically depend on different factors, such as temperature, salt concentration within the solvent, the pH of the sample etc. If removal of a defined amount of solvent is desired, all of these factors have to be carefully considered when calculating the amount of SAP required. In addition, the time needed for reaching the equilibrium state is usually at least 20 minutes, and the method for separating the polymeric material from the residual solvent has to be chosen carefully, in order to not release absorbed water during the separation process.

If, on the other hand, a large excess of superabsorbing material is added to the sample in order to completely absorb the solvent, later on additional steps are necessary for separating the dry target molecules from the swollen polymer.

It was therefore an object of the present invention to provide a method for rapidly concentrating one or more target compounds in a liquid sample, wherein the final sample volume, i.e. the amount of solvent removed from the sample, can be easily adjusted without the need for considering and calculating factors such as pH of the sample solution, salt concentration therein, temperature thereof, and the like.

This has been achieved by the method for the present invention. The present invention provides a method for concentrating one or more target compounds in a liquid sample, said sample comprising said one or more target compounds dissolved in a solvent or a mixture of solvents and having an initial sample volume V₀, the method comprising the step of contacting the sample inside a container with an absorber positioned above the container bottom (vertical position z₀), wherein the final sample volume (V_{f}) is determined by the distance between the lower end of the absorber (vertical position zₐ) and the container bottom (z₀). In other words, the final sample volume is a function (fₓ) of the distance between the lower end of the absorber, i.e. its vertical position zₐ, and the container bottom, V_{f}=fₓ(zₐ—z₀). By positioning an excess of absorber at a certain vertical position zₐ above the container bottom z₀, so that zₐ>z₀, it is possible to obtain a defined final sample volume (or a defined amount of solvent removed from the sample, respectively) in a very short time. Since a large excess of absorber may be used, it is not necessary to wait for establishment of the swelling equilibrium. The volume part of a liquid sample inside the container being present below the lower end of the absorber (zₐ) does not come into contact with the absorber, and thus no solvent is removed from this part of the liquid sample. Accordingly, the solvent will only be removed from that volume part of the liquid sample, which is in contact with the absorber.

In comparison to the methods known from the state of the art, the method and the device of the present invention allow a fast, reliable and reproducible concentration of target compounds in a very simple manner. In addition, the method of the present invention can be easily and economically automated without the need for specifically adapted equipment, using for example standard pipetting devices. In the present invention the amount of solvent removed from a liquid sample can be controlled, e.g. by the level of immersion of a device comprising the SAP into said liquid sample or by the solvent filling level of said sample within a device comprising the SAP, respectively. Accordingly, a large excess of SAP can be used, which in turn means that there is no need to calculate and weigh out a defined amount of SAP for every single sample. There is no need to wait for a swelling equilibrium, either. A precise and reproducible volume reduction can also be achieved in samples of unknown properties, such as ionic strength and pH. In addition, the SAP does not have to be supplied in a perfectly evenly shaped form, but also can be in the form of irregular granules supplied in a basket or a bag, a web or a film coated on the surface of devices, such as tubes, magnetic beads, rods, pipette tips, columns etc.

In terms of the present invention the term “liquid sample” comprises liquids or liquid-containing mixtures of one or more target compounds and a solvent or a mixture of solvents, wherein the target compound is preferably dissolved or suspended in the solvent or the mixture of solvents. The solvent preferably is water or a mixture of water and a water-miscible salts or solvents, such as ethanol, acetonitrile, and the like. The sample may comprise further components, such as non-target molecules, salts, cell debris, and the like, which may also be dissolved in the solvent or mixture of solvents or may be present as sediment.

Preferred target compounds are organic molecules of natural, semi-synthetic or synthetic origin, more preferably organic macromolecules. Particular preferred target compounds are biological polymers. These biomolecules preferably are selected from the group comprising DNA, RNA, peptides, proteins, polysaccharides, and polypeptides, or a mixture thereof, and most preferably represent nucleic acids. Preferred peptides and proteins include enzymes, antibodies, blood-clotting factors, insulin, interferons, hormones, cytokines, transcription factors and other regulatory proteins, or fragments thereof, or a mixture thereof. Nucleic acids (DNA and RNA) may be single-stranded or double-stranded, high molecular weight or short molecules like miRNA, sRNA or highly degraded nucleic acids, and preferably represent viral, bacterial, fungal or cellular RNA, PCR products, genomic, viral, bacterial, fungal or plasmid DNA or cDNA. Further target compounds comprise supramolecular structures, such as viral particles, for example adenovirus, adeno-associated virus, retrovirus, lentivirus, poxvirus, HIV or herpes virus, pro- or eucariotic cells. The aforementioned biological target compounds may be harvested from natural or biotechnical engineered sources, or may be synthesized by recombinant techniques, chemical synthesis, and the like.

As described above, the liquid sample may represent any sample comprising one or more target compounds dissolved or suspended in a solvent or a mixture of solvents. Preferred samples in terms of the present invention are obtained from human, animal or plant tissues, cell cultures, tissue cultures, bone marrow, human or animal body fluids such as blood, serum, plasma, urine, sperm, cerebro-spinal fluids, sputum, swaps, human or animal faeces, plants, plant-parts and extracts, prokaryotic or eukaryotic microorganisms such as bacteria, fungi, viruses, soil samples, mud, waste water, drinking water, or food.

The sample may be used as harvested from its origin or may have been processed prior to employing the method of the present invention, e.g. by cell lysis, removal of cellular material or debris, for example by centrifugation, chromatography, and the like. Especially preferred are diluted solutions of previously purified biological target molecules, e.g. eluates obtained with commonly used chromatographic, spin column, or magnetic bead-based purification protocols and products.

There are different ways of carrying out the present invention. In one preferred embodiment the absorber is comprised in or attached to a device, which is at least partially immersed into the liquid sample inside the container. In this embodiment the absorber preferably is comprised in a hollow object that is at least partially permeable for the solvent, preferably in form of a permeable bag (a principle which is known from tea bags), a permeable basket, or a column with a permeable lower end, wherein said permeable lower end preferably is in the form of a perforated sieve plate, a membrane, a frit, a filter, gauze, or a nozzle. It is not necessary and particularly not preferred that the permeable bag or the membrane is one excluding macromolecules of a specific size to pass, like it is e.g. with an ultrafiltration or dialyze membrane having a size exclusion limit. Thus, in a preferred embodiment the permeable bag or the membrane have no specific compound-selective or size-selective properties. In fact the liquid permeable bag or membrane is preferably only used to keep the absorbent material in a separable means or device, but may be non-selectively permeable for any solved compounds and ingredients of the liquid sample.

The absorber may also be immersed in the liquid sample in form of a device having attached the absorber to at least a part of such surfaces, which are exposed to the solvent, when the device is immersed into the liquid sample, the device preferably being in form of a dip stick, a pipette tip or a rod, at least partially coated with the absorber. The coating preferably is not covered by any compound-selective or size-selective material, like e.g. an ultrafiltration or dialyze membrane. Particularly preferred the coating is not covered by any other material. Other shapes are possible and within the scope of the invention. The device or coating of the device may be structured in such a way as to provide a larger surface for faster absorption.

These embodiments are particularly suitable for automated liquid handling systems, such as an automated pipetting apparatus. Here the final sample volume (V_{f}) can be easily controlled by programming the z-height (vertical position) of the automated handler. The handler then positions the device comprising the absorber or having it attached to its inner or outer surface at a certain height inside an individual sample thus initiating volume reduction. The final sample volume (V_{f}) after concentrating is determined by the distance between the lower end of the absorber (vertical position zₐ) and the container bottom (vertical z₀). FIG. 1 shows a schematic representation of such a device in form of a pipette tip having attached to the lower end of its surface a layer of the absorber.

The final sample volume is a function of the distance between the lower end of the absorber (vertical position zₐ) and the container bottom (vertical position z₀), V_{f}=fₓ(zₐ−z₀). The exact relation including the function fₓ depends upon the dimension and shape of the container, which is well-known to a person skilled in the art. If for example the container has a cylindrical shape with a flat container bottom, such as illustrated in FIG. 2, having a radius r and a height h, wherein the height is equal to z₀ at the container bottom, the final sample volume can be calculated according to the following equation: V_{f}=πr² (zₐ−z₀). Equations for calculating the final sample volume for containers of different shape are known to a skilled person as well.

In another preferred embodiment the liquid sample to be concentrated is filled into a container, said container already comprising the absorber positioned at a certain height (zₐ) above the container bottom (z₀), the absorber preferably being attached to one or more areas of the container's inner surface, wherein the lower end of said coated areas is above the container bottom (zₐ>z₀). The absorber may also be comprised in or attached to a device positioned at a certain height above the container bottom (z₀), wherein the device may already be comprised in the container when the liquid sample is filled in. In this embodiment the device may be integrated into the container in a (re)movable or in a non-removable manner.

If the absorber is attached to the container's inner surface, one continuous area of the inner surface may be coated with the absorber or several isolated areas of the inner surface may be coated with the absorber. The coating itself is preferably not covered by any further material, e.g. a membrane. One example of this embodiment is shown in FIG. 3, showing a so-called Eppendorff tube with two or more isolated areas or a continuous ring-shaped area of the tube's inner surface coated with the absorber.

FIG. 4 shows a device comprising the absorber integrated into the sample container in a removable manner from a side view (left) and a top view (right). This embodiment will be discussed in detail below.

Using the method of the present invention it is possible to use a large excess of absorber with respect to the sample volume to be removed. Accordingly, it is not necessary to wait until a swelling equilibrium is reached, which significantly accelerates the process of concentrating in comparison to the methods known from the state of the art using absorbers. The time needed for concentrating the liquid sample from the initial sample volume (V₀) to the defined final sample volume (V_{f}) is less than 45 minutes, preferably less than 30 minutes, more preferably less than 20 minutes, even more preferably less than 10 minutes, and most preferably is 5 minutes, 1 minute, 30 seconds or less.

The volume reduction, expressed as the ratio of the initial sample volume to the final sample volume (V₀/V_{f}), that can be achieved using the method of the present invention can be chosen in a wide range, depending inter alia upon the amount and kind of absorber used. It is, however, preferred, that V₀/V_{f} is in the range of 2/1 to 50/1, preferably 4/1 to 30/1, more preferably 5/1 to 20/1, and most preferably 8/1 to 12/1.

The absorber preferably comprises a hydrophilic polymer or copolymer, said polymer or copolymer being capable of retaining an amount of water and/or aqueous solutions within its structure by swelling in water and/or aqueous solutions without dissolving. The ratio of the amount of water or aqueous solutions retained within the absorber to the amount of dry absorber material [given in g/g] preferably is at least 2/1, more preferably at least 5/1, even more preferably 20/1, and most preferably at least 50/1. The ability of absorbing and retaining water and/or aqueous solutions depends upon various factors, which is well-known to a person skilled in the art. A crucial role plays the absorber itself, i.e. its chemical constitution, structure, cross-linking, and the like. A further important factor is the nature of the solvent. For example, many SAPs absorb water or aqueous solutions by forming hydrogen bonds with the water molecules. The SAP's ability to absorb water accordingly is a factor of the ion strength in the aqueous solution. In deionized and distilled water, SAPs usually are able to absorb more than a hundredfold their own weight, but in e.g. a 0.9% saline solution, the absorbency often dramatically drops due to the presence of cations (Na⁺) in the solution which impede the polymer's ability of retaining water molecules by hydrogen bonding. Other important factors influencing a polymer's absorbency are environmental conditions, such as temperature, pressure, and the like.

The values for the ratio of the amount of water or aqueous solution retained within the absorber to the amount of dry absorber material [in g/g] given above preferably relate to the ability of absorbing water from a 0.9% saline solution at room temperature (23° C.±2° C.). These values are usually provided by the manufacturer of the absorbing materials, but can also be easily determined by a person skilled in the art.

The hydrophilic polymer or copolymer preferably comprises an organic polymer or copolymer. These organic polymers or copolymers preferably comprise polymerized vinylic monomers and have anionic, cationic and/or zwitterionic side-chains, or a mixture thereof. In a particular preferred embodiment, the hydrophilic polymer or copolymer comprises vinylic monomers and anionic side-chains.

In terms of the present invention a vinylic monomer is any low-weight organic compound (molecular weight less than or equal to 300 g/mol), comprising at least one —CH═CH₂ group. When polymerizing such vinylic monomers, their C═C double bonds react with one another, forming the backbone of the polymer/copolymer. Depending upon the monomers, cross-linkers and/or co-monomers used, the polymer may comprise anionic, cationic and/or zwitterionic side chains. In terms of the present invention anionic side chains are preferred.

Particularly preferred are anionic groups which depending upon the pH may be present in either a protonated or a deprotonated state (for example carboxylate groups —CO₂H/—CO₂⁻). Such groups are able to form hydrogen bonds with water molecules and thus effect hydration of the absorber. It is particularly preferred, that these side chains are at least partially neutralized, i.e. by sodium hydroxide, so that in the dry (non-swollen) gel a certain amount of side chains is present in a charged state, wherein the charged groups repel each other. Overall electrical neutrality of the dry polymer is maintained since the negative groups are balanced by the positive counterions originating from the base used for neutralizing. Upon contact with water these counterions become hydrated, which reduces the attraction to the oppositely charged ionic side-chains due to the high dielectric constant of water. The hydrated counterions are then able to freely move within the polymeric network, contributing to the osmotic pressure inside the gel. As the counterions are, however, still weakly attracted by the oppositely charged groups in the polymer backbone, they do not leave the gel, but are a kind of entrapped. The resulting difference between the osmotic pressure inside and outside the gel is the driving force for swelling. Increasing the level of sodium outside the gel will accordingly lower the osmotic pressure and hence reduce swelling capacity of the gel.

The vinylic monomers preferably are acrylic monomers, more preferably selected from the group comprising acrylic acids, methacrylic acids, acrylates, methacrylates, acrylonitriles, acrylamides and methacrylamides, or mixtures thereof.

Preferred absorbing polymers or copolymers are so-called superabsorbing polymers, which in comparison to “classic” absorbing materials known from the state of the art, e.g. diapers or tissue paper, have the ability to absorb and retain extraordinary large amounts of water or aqueous solutions. In addition, SAPs are able to preserve water after absorption and swelling. This ability, expressed as swollen gel strength, discriminates SAPs from other hydrogels or traditional absorbing materials such as tissue papers or cotton boards, which loose a large amount of absorbed water upon squeezing.

Typical parameters characterizing a superabsorbing polymer are the absorbency under load (AUL), the swelling rate, the swollen gel strength, the soluble fraction, and the ionic sensitivity, which can be determined as described in the review article by M. J. Zohuriaan-Mehr and K. Kabiri Iranian Polymer Journal, 2008, 17 (6), 451-477.

The absorbency under load usually implies the uptake of water or an 0.9% saline solution while the testing sample is pressurized by a specified load.

The swelling rate is determined by measuring remaining volumes after certain time points.

The soluble fraction (sol) is the sum of all water-soluble species in the absorber, including non-crosslinked oligomers and unreacted starting material, i.e. residual monomers, that may be released into solution. The soluble fraction can be easily determined by extracting a sample of the absorbing material using distilled water, filtering the swollen sample and weighting the sample after oven-drying, the loss of sample weight representing the soluble fraction.

In terms of the present invention the use of absorbers is preferred, which show a low absorbency-loss in salt solutions in comparison to deionized and distilled water. The dimensionless swelling factor sf (sf=1−(absorption in a given fluid/absorption in distilled water)) provides a comparative measure of the sensitivity of the absorbing materials towards the kind of aqueous fluid.

The absorber used in the method of the present invention may be an organic polymer or copolymer, preferably a cross-linked polymer, which comprises one or more types of cross-linkers, preferably selected from the group comprising N,N′-methylenebisacrylamide, N,N′-ethylenebisacrylamide, 1,3,5-triacroyl hexahydro-1,3,5-triazine (THHT), pentaerythritol tetraacrylate (PETA), trimethylolpropane triacrylate (TMPTA), and diethyleneglycol diacrylate, or mixtures thereof.

By cross-linking the polymer chains a three-dimensional network in the absorber is obtained which prevents the polymer from swelling to infinity by its elastic retraction forces. The degree of cross-linking in an absorbing polymer has influence on the swelling capacity as well as to the absorbency under load. While a highly cross-linked polymer has a low swelling capacity, a very low cross-linked polymer exhibits a high swelling capacity, but a poor absorbency under load, i.e. the polymer will lose most of the absorbed water under pressure. The preferred amount of cross-linker in the polymer is in the range of 0.01 to 5 wt-% especially preferred in the range of 0.1 to 1 wt-%.

By using a combination of different monomers or a combination of two or more different polymer system in the form of interpenetrating networks it is possible to fine-tune the desired properties of the SAP.

Thus in a preferred embodiment the polymer or copolymer comprises at least one type of vinylic monomers, preferably at least acrylic monomers, and preferably one or more additional types of monomers, preferably selected from the group comprising e.g. acrylic acid, acrylic acid anhydride, hydroxyethyl(met)acrylate, glycidyl(met)acrylate.

In another preferred embodiment the polymer is a copolymer system, comprising at least one acrylic polymer and one or more additional polymer(s) selected from the group comprising polyethylene glycols (PEG) and polysaccharides. The one or more additional polymer(s) preferably are polymers selected from the group comprising PEG, dextrose, agarose, and chitosan, or mixtures thereof. Particular preferred copolymer systems with advanced properties are acrylics-chitosan copolymer systems, which exhibit high swelling under acidic conditions, acrylics-PEG copolymer systems, wherein the mesh width can be easily controlled by lateral chain aggregation and acrylics-agarose copolymer systems, having an enhanced stability in comparison to pure acrylic polymer systems.

Upon hydration of the absorber a three-dimensional network is formed, having a certain mesh width and a specific mesh pattern, defined by the mean diameter of the mesh and its geometrical form. The geometrical form and the mesh size define the molecular cut-off properties of the meshes, usually given in kilo Dalton (kDa) or base pairs (bp) for macromolecules. The molecular cut-off defines the upper size limit, above which molecules are too large to enter the polymer network and thus cannot be entrapped by it. The molecular cut-off value can be controlled by the type and amount of cross-linker in the polymer. The mesh width (molecular cut-off) inside the gel, formed by the hydrophilic polymer or copolymer upon swelling in the solvent. It is known to a person skilled in the art that the mesh width varies in time during exposure to the aqueous media due to the transition from a coiled to an expanded state, until a maximum is reached. The aforementioned values for the preferred mesh width refer to the maximum mesh width. The mesh width chosen in a particular concentrating process depends upon the target compound to be concentrated. Preferably, the mesh width is lower than the size of the target compounds, so that the target compounds cannot enter the absorbing polymer, even if the hydrated network is fully expanded.

Preferred target compounds represent biomolecules, more preferably selected from the group comprising DNA, RNA, peptides, proteins, polysaccharides, and polyketides, and most preferably represent DNA and RNA, with sizes between about 18-20 up to hundreds or thousands of nucleotides.

Accordingly the mesh width (molecular cut-off) of the absorbing material may be e.g. from 500, 1.000, 5.000, 10.000, 20.000, 30.000, 40.000 or 50.000 to 100.000, 150.000, 200.000, 250.000 or 300.000 Dalton, dependent from the molecular weight of the target molecules, or may be such that oligonucleic acids having e.g. from 10, 15, 20, 30, 100 by or nucleotides, or polynucleic acids having more than 100, 250, 500 or 1.000 and e.g. up to 3.000, 5.000, 10.000, 25.000 or 50.000 by or nucleotides cannot pass into the absorbing material.

In terms of the present invention the SAP may be in the form of particles, such as non-regular granules or spherical beads, fibers, webs, films, surface coatings, and the like.

To further enhance the swelling rate of the SAP and its solvent retaining properties under pressure, for example during centrifugation, the surface or shell of particles, fibers or films may be specifically cross-linked. After a first polymerization step to form the polymeric network of the SAP, a cross-linking solution is then applied to the dried SAP in form of particles, fibers, or films (so-called post cross-linking). As a cross-linker, molecules possessing at least two functional groups capable of reacting with the functional groups of the side-chains (for example the carboxyl groups in case of polyacrylic polymers) are used, for example polyalcohols such as glycerin. This post cross-linking increases the density of cross-linking on the surface of the beads, fiber, films etc. leading to a kind of core-shell particle, fiber or film. While the core of such a particle, fiber or film consists of a lightly cross-linked polymer, the shell has a higher cross-linking density on its surface.

The polymer may be further modified by coating the absorber's surface with different polymers in order to tune the absorber's properties, e.g. for enhancing biocompatibility or for minimizing interactions between the target compounds and the absorber. In a particular preferred embodiment the absorber's surface is modified to minimize any attracting interactions between the target compounds and the absorber, preferably by (i) selectively cross-linking the absorber surface after polymerization, (ii) covalently attaching further molecules to the absorber surface and/or (iii) coating the absorber surface with an additional polymer, said additional polymer preferably being selected from the group comprising polyethylene glycol (PEG), polyethyleneimine (PEI), polylysine (PL), and polyvinylpyrrolidone (PVP), or mixtures thereof.

Further parameters that may influence the water-adsorption capacity of the polymer are the temperature (preferably room temperature), the pH of the samples to be concentrated (preferably near neutral or slightly basic, i.e. 6-9, 7-8.5), the concentration of the target molecules in the sample before concentration, the content of salts (ionic strength) in the sample solution (0-200 mM), and the average molecular weight of the absorbing polymers.

If the absorber is provided in the form of spherical beads, the diameter preferably is in the range of 0.1-1 mm, more preferred 0.2-0.4 mm. If the absorber is used in form of a film or web, the-film or web preferably has a layer thickness in the range of 0.01-1 mm, more preferred 0.1-0.4 mm. If the target molecule includes nucleic acids, it is preferred to use an absorbing polymer which is free of any alginate and nucleases.

The invention further provides a device particularly adapted for concentrating one or more target compounds in a liquid sample inside a container according to the method described above, wherein the device as exemplified in FIG. 4 comprises a hollow body 4 with a permeable bottom end 6 and an upper end 5, which can be non-permeable, the hollow body 4 comprising an absorber 3 as described above and optionally means 7 for reversibly fixing the device at a certain height zₐ inside the container 2. A non-permeable upper end 5 preferably is in the form of a sloping non-permeable upper end, and the permeable bottom end 6 preferably is in the form of a perforated sieve plate, a membrane, preferably a membrane which is non-selective for the solved ingredients and in particular non-selective for the target compounds, a frit, a filter, gauze, or a nozzle. Such device can be integrated into a container 2, e.g. a collection tube or multi-well plate, in a removable manner and can be easily fixed at a certain height zₐ inside the container by means 7. The device can be placed in an empty container and fixed at a certain height zₐ that determines the final sample volume V_{f} after concentrating. The liquid sample is then filled into the container, whereby it passes by the device without contacting the absorber and is collected at the bottom of the container. In this embodiment, a sloping non-permeable upper end is preferred to ensure that the liquid sample rapidly passes by the device. As well the liquid sample can be filled into the container before the device is placed in both the container and the liquid. The lower end and/or the sides of the device is permeable, for example in the form of a perforated sieve plate, a membrane, preferably a membrane which is non-selective for the solved ingredients and in particular non-selective for the target compounds, a frit, a filter, or a nozzle. As soon as the filling level inside the container reaches the lower end of the device, the solvent present above this level will be absorbed by the absorbing material inside the device. Since the device can be easily removed from the sample/the container comprising the sample, it is possible to use a very large excess of the absorber. By selecting the amount and type of absorber comprised within the device, it is possible to create a device that minimizes time loss during volume reduction, i.e. virtually at the same time the process of filling the sample into the container is finished, volume reduction is finished, too. The absorbed solvent, being trapped in the absorber inside the device, can be easily removed from the sample container by removing the whole device, and can later be discarded.

The device furthermore may represent a dipstick, a pipette tip, a column or a rod, at least partially coated with the absorber. The device may also represent a bag, a basket or other container filled with the absorber and being permeable for the solvent. It is particularly preferred that the material surrounding the absorber—if present—is essentially non-selective for the solved ingredients contained in the liquid sample and in particular non-selective for the target compounds. In a further embodiment, the device may represent a container whose inner surface is partially coated with the absorber and wherein the lower end of said coated area is above the container bottom (zₐ), i.e. zₐ>z₀.

Independent from the embodiment of the device comprising the absorber according to the invention it is preferred that said device comprises a means for—preferably reversibly—positioning or fixing the device in a container in a way that the lower end of said device (zₐ) is spaced above the container bottom, i.e. zₐ>z₀. Preferably the space between the lower end of the absorbing device and the bottom end of the container results in a defined volume, particularly a defined volume of a liquid when contained in this area.

Further according to the invention a container is provided comprising any of the above mentioned embodiments of the device comprising the absorber, wherein said container further comprises a means for positioning or fixing said device—preferably reversibly—inside of the container at a determined space (zₐ) above the container bottom, i.e. zₐ>z₀. Preferably the space between the lower end of the absorbing device and the bottom end of the container results in a defined volume, particularly a defined volume of a liquid when contained in this area.

Due to the preferred use of a crosslinked polymeric absorber material it is not necessary and not preferred to combine the absorber with a further coating or layer of any material allowing a selection of the ingredients of the liquid sample, like e.g. the provision of an ultrafiltration or a dialyze membrane. The absorber can be used in immediate contact with the liquid sample. With “immediate contact” is meant that the absorber either is not covered by any material, like e.g. a selective or non-selective coating, a selective or non-selective film, a selective or non-selective membrane, a selective or non-selective layer or similar on a surface contacted with the liquid sample, or the absorber indeed is contained in a bag, a basket or a container provided with a sieve plate, a frit, a filter, a nozzle or a membrane. However, said bag, basket, sieve plate, frit, filter, nozzle or membrane only serves as a means for keeping the absorber separable from the liquid sample, but allows the liquid sample to pass without any selection of the solved ingredients and in particular without any selection of the target compounds.

The method and/or the device of the present invention can be used for concentrating one or more target compounds in a biological sample. Preferably the method and/or the device preferably e.g. can be used for concentrating pathogens or nucleic acid in body fluids, said body fluids preferably being selected from the group comprising urine and blood plasma or serum; used for concentrating prokaryotic or eukaryotic cells in fluids like body fluids, media, solutions, or liquid environmental samples; for concentrating nucleic acids, preferably RNA, in an eluate obtained after having chromatographically purified or while chromatographically purifying the biological sample; e.g. using spin, gravity flow, or positive pressure columns, magnetic beads, or other commonly used formats; for concentrating free-circulating RNA and/or DNA in blood samples, plasma or serum or for concentrating free-circulating fetal RNA and/or DNA in the maternal plasma or serum; used for concentrating molecular targets like nucleic acid, proteins, carbohydrates or any other larger molecule of interest in liquid environmental samples, e.g. water samples. As the method and the device of the present invention can be used to rapidly and reliably concentrate biological target compounds, it may be used as well in molecular infection or tumor diagnosis (e.g. detection of free circulating tumor DNA or RNA in blood plasma).

The present invention further provides a kit for processing a biological liquid sample comprising a device according to the present invention and further components selected from the group comprising buffers, liquid reactants or reagents, lyophilized enzymes or reagents, plastic consumables optimized for the procedure (e.g. supporting/holding the concentrator device, sample tubes or plates) and chromatographic columns for the purification of one or more target compounds in the sample, or a mixture thereof.

The device comprised in the kit preferably comprises a hollow body 4 with a non-permeable upper end 5 and a permeable bottom end 6, preferably in form of a nozzle, the hollow body 4 comprising an absorber 3 and optionally means 7 for reversibly fixing the device at a certain height zₐ inside a collection tube.

In another preferred embodiment the kit comprises a container, preferably a collection tube, whose inner surface is partially coated with the absorber and the lower end of said coated area(s) is above the container bottom z₀, i.e. zₐ>z₀.

### FIGURES

FIG. 1 exemplarily illustrates one embodiment of the method and device of the present invention, wherein a pipetting tip, coated on its outside surface with the absorber 3, is immersed into a sample collection vessel (container) 2, comprising a liquid sample 1 at a certain height zₐ. After concentrating, all solvent previously present in the vessel above the height zₐ is removed from the liquid sample by the absorber 3.

FIG. 2 schematically shows a container of cylindrical shape with a height h and a radius r, the flat container bottom being at a height z₀. The desired liquid volume can be obtained by introducing the absorber into the container down to height zₐ.

FIG. 3 shows another embodiment of a device and method for carrying out the present invention, wherein the absorber 3 is coated on the inner surface of a sample tube 2 at a certain height. A liquid sample 1 is filled in said container 2, whereupon all solvent present above the lower end zₐ of the absorber 3 is removed from the sample by swelling of the absorber.

FIG. 4 shows a device comprising a hollow body 4 with a sloping non-permeable upper end 5 and a permeable bottom end 6 in form of a nozzle, comprising an absorber 3 and means 7 for reversibly fixing the device at a certain height zₐ inside a container 6.

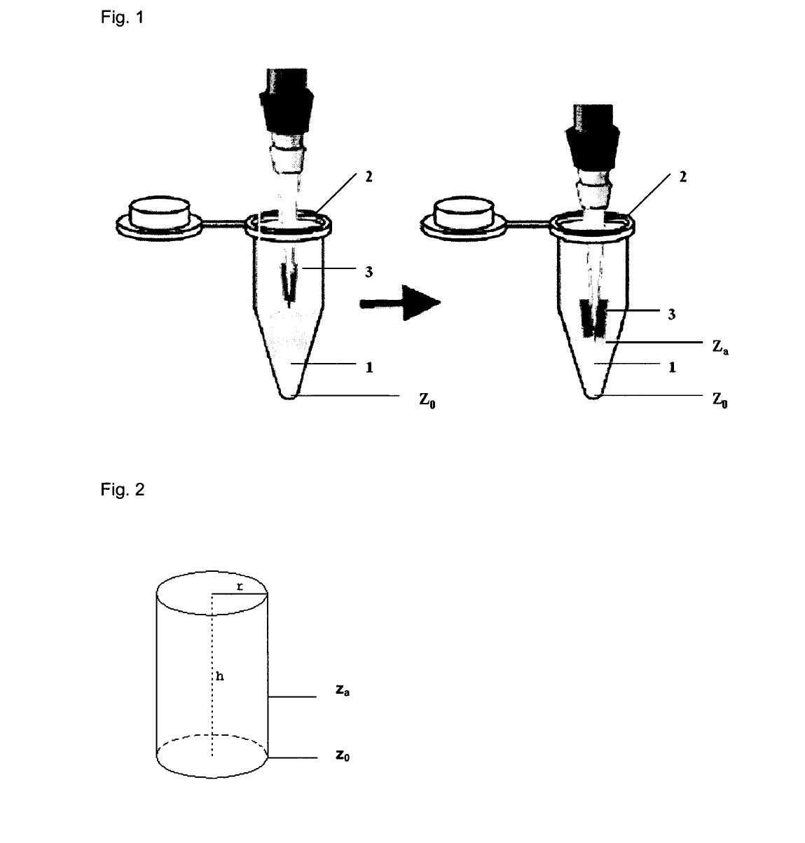

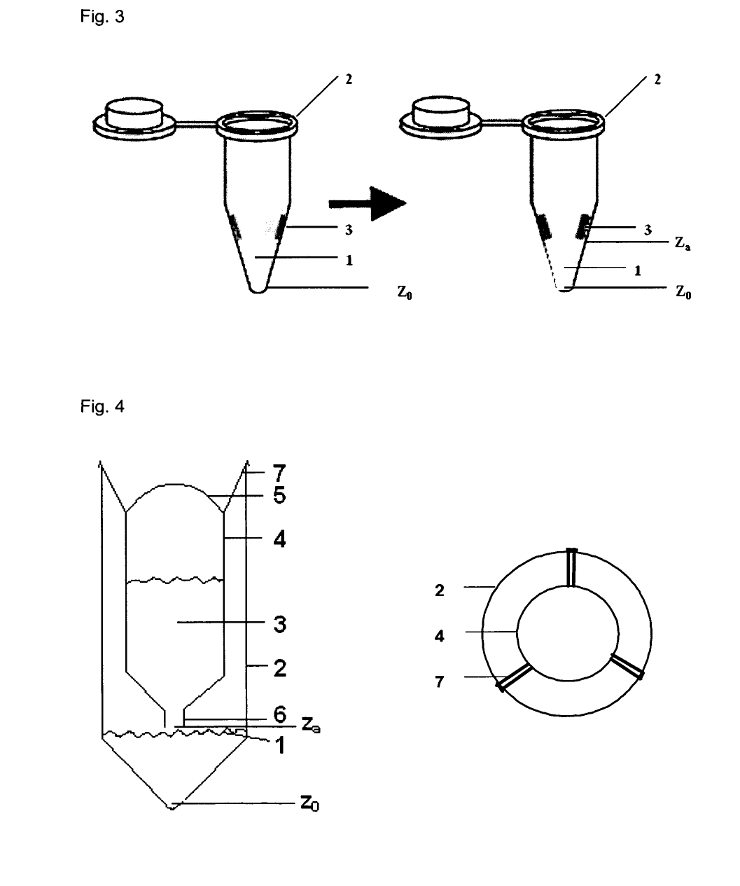

1.-17. (canceled)

18. A method for concentrating one or more target compounds in a liquid sample inside a container, wherein the liquid sample comprises said one or more target compounds dissolved in a solvent or a mixture of solvents and has an initial sample volume V₀, the method comprising:
(A) contacting the sample inside a container with an absorber positioned above the container bottom (z₀), wherein the final sample volume (V_{f}) is determined by the distance between the lower end of the absorber (vertical position zₐ) and the container bottom (z₀).

19. The method of claim 18, wherein the absorber is comprised in or attached to a device that is at least partially immersed into the liquid sample inside the container.

20. The method of claim 19, wherein said device
(a) is in form of a hollow object that comprises the absorber and is at least partially permeable for the solvent, or
(b) has the absorber attached to at least a part of its (inner or outer) surfaces that are exposed to the solvent when the device is immersed into the liquid sample.

21. The method of claim 20, wherein the device of (a) is in form of a permeable bag, a permeable basket, or a column with a permeable lower end.

22. The method of claim 21, wherein said permeable end is in form of a perforated sieve plate, a membrane, a frit, a filter, gauze, or a nozzle.

23. The method of claim 20, wherein the device of (b) is a dip stick or a rod that is at least partially coated with the absorber.

24. The method of claim 18, wherein the liquid sample is filled into the container that already contains the absorber positioned at a certain height above the container bottom (z₀).

25. The method of claim 24, wherein the absorber
(a) is attached to one or more areas of the container's inner surface, wherein the lower end zₐ of said coated areas is above the container's bottom (zₐ>z₀), or
(b) is comprised in or attached to a device positioned above the container bottom (z₀).

26. The method of claim 19, wherein the time needed for concentrating the liquid sample from the initial sample volume (V₀) to the defined final sample volume (V_{f}) is less than 45 minutes, less than 30 minutes, less than 20 minutes, less than 10 minutes, or 5 minutes or less.

27. The method of claim 19, wherein the ratio of the initial sample volume to the final sample volume (V₀/V_{f}) is in the range of 2/1 to 50/1, 4/1 to 30/1, 5/1 to 20/1, or 8/1 to 12/1.

28. The method of claim 19, wherein the absorber comprises a hydrophilic polymer or copolymer capable of retaining an amount of water and/or aqueous solution within its structure by swelling in water and/or aqueous solution without dissolving.

29. The method of claim 28, wherein the ratio of the amount of water or aqueous solution retained within the absorber to the amount of dry absorber material [in g/g] is at least 2/1, at least 5/1, at least 20/1, or at least 50/1.

30. The method of claim 28, wherein the hydrophilic polymer or copolymer comprises an organic polymer or copolymer.

31. The method of claim 30, wherein the organic polymer or copolymer comprises polymerised vinylic monomers and has anionic, cationic and/or zwitterionic side-chains, or a mixture thereof.

32. The method of claim 31, wherein the organic polymer or copolymer has anionic side-chains.

33. The method of claim 31, wherein the vinylic monomers are acrylic monomers.

34. The method of claim 33, wherein the acrylic monomers are selected from the group consisting of acrylic acids, methacrylic acids, acrylates, methacrylates, acrylonitrile, acrylamides, methacrylamides, and mixtures thereof.

35. The method of claim 30, wherein the organic polymer or co-polymer is a cross-linked polymer that comprises one or more types of cross-linkers.

36. The method of claim 35, wherein the cross-linkers are selected from the group consisting of N,N′-methylenebisacrylamide, N,N′-ethylenebisacrylamide, 1,3,5-triacroyl hexahydro-1,3,5-triazine (THHT), pentaerythritol tetraacrylate (PETA), trimethylolpropane triacrylate (TMPTA), diethyleneglycol diacrylate, and mixtures thereof.

37. The method of claim 36, wherein the amount of crosslinker in the polymer is in the range of 0.01 to 5 wt-%.

38. The method of claim 28, wherein the polymer is
(a) a copolymer that comprises a least one type of vinylic monomer and one or more additional types of monomers, or
(b) a copolymer system that comprises at least one type of acrylic polymer and one or more additional polymers selected from the group consisting of polyethyleneglycols (PEG) and polysaccharides.

39. The method of claim 38, wherein the additional polymers of (b) are selected from the group consisting of PEG, dextrose, agarose, chitosan, and mixtures thereof.

40. The method of claim 28, wherein the mesh width (molecular cut-off) inside the gel formed by the hydrophilic polymer or copolymer upon swelling in the solvent is less than 50 nm.

41. The method of claim 40, wherein the mesh width is 10 nm or less.

42. The method of claim 19, wherein the target compounds are biomolecules.

43. The method of claim 42, wherein the biomolecules are selected from the group consisting of DNA, RNA, peptides, proteins, polysaccharides, and polyketides.

44. The method of claim 43, wherein the biomolecules are DNA or RNA.

45. The method of claim 19, wherein the absorber's surface is modified to minimize any attracting interactions between the target compounds and the absorber.

46. The method of claim 45, wherein the absorber's surface is modified to minimize attracting interactions between the target compounds and the absorber.

47. The method of claim 46, wherein the absorber's surface is modified by
(i) selectively cross-linking the absorber's surface after polymerization,
(ii) covalently attaching further molecules to the absorber's surface, and/or
(iii) coating the absorber's surface with an additional polymer.

48. The method of claim 46, wherein the additional polymer of (iii) is selected from the group comprising polyethylenglycol (PEG), polyethylenimine (PEI), polylysine (PL), polyvinylpyrrolidone (PVP), and mixtures thereof.

49. The method of claim 18, wherein the method is used for
(a) concentrating pathogens or nucleic acids in body fluids,
(b) concentrating nucleic acids in an eluate obtained after having chromatographically purified or while chromatographically purifying the biological sample,
(c) concentrating free-circulating RNA and/or DNA in a blood sample,
(d) concentrating free-circulating fetal RNA and/or DNA in the maternal plasma or serum,
(e) concentrating free-circulating tumor RNA and/or DNA in the plasma or serum, or
(f) concentrating molecular targets in liquid environmental samples.

50. The method of claim 49, wherein the eluate of (b) is obtained after having chromatographically purified or while chromatographically purifying the biological sample using spin columns or magnetic beads.

51. The method of claim 49, wherein the body fluids of (a) is selected from the group consisting of urine and blood plasma or serum.

52. The method of claim 49, wherein the nucleic acids of (b) are RNA and/or DNA,

53. The method of claim 49, wherein the molecular targets of (f) are nucleic acid, proteins, carbohydrates, or other larger molecules of interest.

54. The method of claim 53, wherein the liquid environmental samples of (f) are water samples.

55. A device for use in a method for concentrating one or more target compounds in a liquid sample inside a container of claim 19, wherein
(a) said device comprises a hollow body with a non-permeable upper end and a permeable bottom end, said hollow body comprising an absorber and optionally means for reversibly fixing the device at a certain height (zₐ) inside the container;
(b) said device is a dipstick or a rod that is at least partially coated with the absorber;
(c) said device is a bag or a basket filled with the absorber and is permeable for the solvent; or
(d) said device is a container whose inner surface is partially coated with the absorber, wherein the lower end of said coated areas is above the container bottom (z₀).

56. The device of claim 55, wherein said non-permeable upper end of the hollow body in (a) is in form of a sloping non-permeable upper end.

57. The device of claim 55, wherein said permeable bottom end of the hollow body in (a) is in form of a perforated sieve plate, a membrane, a frit, a filter, or a nozzle.

58. A kit for processing a biological sample, comprising:
(i) a device of claim 55, and
(ii) further components selected from the group consisting of buffers, liquid reactants or reagents, lyophilized enzymes or reagents, plastic consumables optimized for the use of the device or for concentrating one or more target compounds in a liquid sample, chromatographic columns for the purification of one or more target compounds in the sample, and a mixture thereof.

59. The kit of claim 58, wherein
(a) the device comprises a hollow body with an optionally non-permeable upper end and a permeable bottom end, said hollow body comprising an absorber and optionally means for reversibly fixing the device at a certain height (zₐ) inside said container; or
(b) the device is a container whose inner surface is partially coated with the absorber, the lower end of said coated areas (zₐ) being above the container bottom (z₀).

60. The kit of claim 59, wherein said permeable bottom end of the hollow body in (a) is in form of a perforated sieve plate, a membrane, a frit, a filter, or a nozzle.
